(19) United States
(12) Patent Application Publication
(10) Pub. No.: US 2009/0324722 A1
(43) Pub. Date: Dec. 31, 2009

(54) CARTILAGE FILLING DEVICE

(76) Inventor: Jennifer H. ELISSEEFF, Baltimore, MD (US)

Correspondence Address: Peter F. Corless;Edwards Angell Palmer & Dodge LLP, P.O. Box 55874, Boston, MA, 02205, US

(21) Appl. No.: 12/414,089

(22) Filed: Mar. 30, 2009

Related U.S. Application Data
(63) Continuation of application No. 11/663,476, now abandoned.
(60) Provisional application No. 60/611,754, filed on Sep. 22, 2004.

(51) Int. Cl. A61K 9/14; C07H 15/00

(52) U.S. Cl. 424/487; 536/123.1

(57) ABSTRACT

Compositions and methods for treating a tissue defect.

### BACKGROUND

Arthritis is a painful deterioration of the cartilage lining ofjoints known as DJD (degenerative joint disease). It affects over 70 million Americans and is responsible for over $86 B/year in costs and lost productivity in the United States (CDC, 2004). Both numbers are projected to go up as the existing adult population matures. To date, the treatments for arthritis have been to minimize and control pain with anti-inflammatory drugs, joint injections of cortisone shots, and the new viscosupplementation of hyaluronic acid. There are roughly 650,000 knee arthroscopies performed per year in the U.S., ostensibly to relieve pain. However, these procedures often delay the inevitable: joint replacement In the United States there are over 500,000 knee and hip replacements per year (700,000 overall). Joint replacements often help with pain relief and restore some joint function, but patients are very limited in what they can and cannot do and their diseased joint and bone is removed permanently. For younger patients (<45 years) activities such as running, playing sports such as basketball, soccer, tennis, squash are discouraged if not forbidden after joint replacements. The hip replacements fail roughly 5% of the time and last roughly 10 years. Revision of joint replacements (second surgery) is a growing concern because people are requiring joint replacements earlier in their lives and wear out the artificial joint by trying to lead active lives.

Scientists working on cartilage growth have met with limited success. For instance, Genzyme has a product/service called Carticel in which doctors must perform one surgery and scrape cartilage cells from a patient's knee, grow it outside the body, and then surgically re-implant the grown tissue into the patient's knee. However, the problems with this process are that the cartilage that is grown is not necessarily long lasting and durable, and the cartilage that is re-implanted in the body may become fibro-cartilage and not the smooth, lubricating hyaline cartilage that covers bone ends around joints in the body. Also, the process is not approved for the treatment of arthritis. In addition, this procedure requires two surgeries. Genzyme also manufactures Synvisc, which is an injectable hyaluronic acid treatment for joints that can provide up to 6 months of pain relief from a treatment of usually 3 intrajoint injections.

Other current treatment modalities include that provided by Biosyntech, Montreal, Canada. Their product is BST-Cargel. They mix the patient's blood with a chitosan gel that is thermally activated in the body. They then drill into the bone marrow to harvest pluripotent stem cells that are mixed with the gel.

Histogenics Corporation is a tissue engineering company. The company combines device technology and tissue engineering to streamline methodologies for exogenous cell and tissue growth. The proprietary Tissue Engineering Support System (TESS) is used to grow stable cell matrices (NeoCart), of patient cartilage tissue. The tissue is then surgically inserted back into the patient. TESS consists of two primary elements (1) a matrix that supports cells seeded into it, promotes healthy growth and histogenesis of those cells, and eventual in vivo integration of the healthy neo-tissue, and (2) a processor for providing the optimum environment for the target histogenesis. In addition to being entirely self-contained, the processor allows all significant parameters for tissue growth and development to be computer controlled in real-time. Unique to TESS, is the ability to deliver and control hydrostatic fluid pressure. That characteristic is important to the development of cartilage and other tissue that readily acquires mature morphology with pressure.

Osiris focuses in the mesenchymal stem cell area including growing tissue such as cartilage in a gel.

OsteoBiologics is a bone/cartilage repair company using synthetic/ceramic materials.

Geron has a stake in the embryonic stem cell arena. The company plans to inject embryonic stem cells into joints to grow new cartilage.

Arthrex has a treatment for arthritis where blood from a patient is filtered and the purified blood in introduced into the affected joint offering up to 6 months of pain relief.

Zimmer has the Hedrocel® biomaterial marketed as Trabecular Metal™ for implant bone regrowth. They also have a process where “neo-cartilage” is grown from young cadaver cartilage that is seeded with chondrocytes.

3DM markets Puramatrix that is a hydrogel that can be used to form scaffolds.

ACRU (Articular Cartilage Repair Unit) is a part of Depuy that uses a resorbable, acellular polyglycolic acid (PGA)/polylactic acid ALA) copolymer that is seeded by marrow cells. The Depuy Mitek Autograft Implantation System (CAIS) product is a resorbable, copolymer, resurfacing scaffold (polydioxanone (PDS) mesh with PGA/PCL foam) that uses an arthroscopic harvest methodology to re-implant articular cartilage cells.

Exactech, Inc., distributes Opteform, a bone allograft material, under a distribution agreement with the University of Florida Tissue Bank.

Salumedica, Inc. markets SaluCartilage, designed to be a less invasive solution to pain and immobility due to cartilage defects as a result of arthritis and sports injury. SaluCartilage is a synthetic implant developed to replace worn-out cartilage surfaces, restoring mobility and relieving joint pain. The damaged articular cartilage is cored out and replaced with SaluCartilage to provide a smooth, load-bearing joint surface. The implant is not biodegradable.

Fidia manufactures Hyalgan, a viscosupplementation product of hyaluroic acid that is injected into arthritic joints to provide pain relief and increase joint mobility.

Cortisone injections can provide a few months of pain relief.

However, none of the above technologies slow down or reverse the arthritis process as do injections of hyaluronic acid. Growth hormone injections into the joint have been posited to initiate growth. Surgery to repair torn and damaged cartilage, including microfracture surgery in which a surgeon intentionally cuts into the bone around damaged tissue to promote new bone/cartilage growth has been proposed as treatments.

### SUMMARY OF THE INVENTION

The instant invention relates in part to a unique gel in which hyaline cartilage can develop either from autologous stem cells or develop per se from extant chondrocytes.

The instant invention also relates to a procedure that is minimally invasive (potentially arthroscopic and <1 hour of surgery as compared to several hours for a joint replacement), will be outpatient, and will require far less physical rehabilitation, 3-4 weeks vs. months. There is the potential to return to the same level of activity prior to the injury or onset of arthritis. These factors are of great benefit to patients especially younger patients who may prematurely develop arthritis and are not candidates for joint replacement surgeries because of age or a desire to remain athletic and participate in joint stressing activities or sports.

The invention provides for novel compounds and compositions, such as tissue adhesives which secure the hydrogel to a cartilage surface comprising a polymer that contains at least two functional groups, one which reacts with functional groups found in cartilage or bone, and the other which is reactive with the hydrogel. An additional composition of interest is the hydrogel/primer complex.

The instant invention also relates to a kit comprising the materials for treating the cartilage defect, as well as optionally, a device for microfracture of the adjacent bone. Alternatively, a kit of interest comprises a doped hydrogel, a patch to hold the gel in place, and a UV source to photopolymerize the gel.

### DETAILED DESCRIPTION

The instant invention relates to a method for filling or finishing a cartilage defect. The method comprises applying to the cartilage surface a hydrogel. Optionally, the cartilage surface can first be treated with a primer that attaches to the cartilage surface and reacts with the hydrogel. Optionally, the cartilage defect can be covered with a film that serves as a mold for the pregelled hydrogel solution. Optionally, the osseous regions in the vicinity of the cartilage defect can be microfractured. The microfracture can be obtained by inserting a suitable device through the hydrogel, or the microfracture can occur by using a device that effects the microfracture from the side of the bone distal from the cartilage defect.

Gels and films of interest encourage the growth of hyaline cartilage in the presence of stem cells, addressing one of the major problems with stem cell based therapies—controlling the expression of the stem cells. The instant invention accomplishes that task.

In vitro, the tissue-engineering product, for example, a photopolymerizable hydrogel, such as PEODA (polyethylene oxide diacrylate), can be used to encapsulate mesenchymal stern cell (MSC) to support their survival and chondrogenic differentiation. In a subcutaneous mouse model, a blend of PEODA and high molecular weight hyaluronic acid (HA) was mixed with MSCs, injected under the skin and polymerized transdermally. MSC chondrogenesis and cartilage tissue formation was further enhanced by the presence of HA in larger animal models

Significant to a product of interest is the enhanced integration with the surrounding cartilage to increase implant stability and bonding of newly formed tissue. In vitro studies have proven their efficacy by showing the chemical mechanism of reacting to the cartilage surface and the increased mechanical strength of the material-cartilage interface. Magnetic resonance (MR) was used to quantifiably define the hydrogel stability and bonding to cartilage when using the integration method of choice. MR was used to measure hydrogel volume retained in the defect, and to determine the extent of integration with native cartilage.

The instant invention addresses the problem of fibrocartilage formation in any of the above stated surgical methods and yet enables a combination of marrow stimulation and cell transfer in a minimally invasive manner. The instant invention is also usable in early osteoarthritic joints by using patches and gels to prevent enzymatic synovial degradation during and after implantation. The instant invention also enables marrow stimulation without disrupting subchondral bone integrity. Finally, the instant invention enables the use of computer assisted surgical navigation to increase the accuracy of surgical implantation in a minimally invasive manner.

The instant invention provides for in situ polymerization techniques to form hydrogel scaffolds that can be molded to take the desired shape of the defect, promote tissue development by stimulating native cell repair, and can be potentially implanted by minimally invasive injection.

The terms “active agent,” and “biologically active agent” are used interchangeably herein to refer a chemical or biological compound that induces a desired pharmacological, physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent” and “drug” are used, then, it is to be understood that applicants intend to include the active agent per se as well as pharmaceutically acceptable, pharmacologically active salts, esters, amides, prodrugs, metabolites, analogs etc.

The terms “biocompatible polymer”, “biocompatible cross-linked polymer matrix” and “biocompatibility” when used in relation to polymers are art-recognized. For example, biocompatible polymers include polymers that are neither toxic to the host (e.g., an animal or human), nor degrade (if the polymer degrades) at a rate that produces monomeric or oligomeric subunits or other byproducts at toxic concentrations in the host.

In certain embodiments of the present invention, biodegradation generally involves degradation of the polymer in an organism, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions be biocompatible as set forth above. Hence, a subject composition may comprise polymers comprising 99%, 98%, 97%, 96%, 95%, 90%, 85%, 80%, 75% or even less of biocompatible polymers, e.g., including polymers and other materials and excipients described herein, and still be biocompatible.

To determine whether a polymer or other material is biocompatible, it may be necessary to conduct a toxicity analysis. Such assays are well known in the art. One example of such an assay may be performed with live carcinoma cells, such as GT3TKB tumor cells, in the following manner: the sample is degraded in 1M NaOH at 37° C. until complete degradation is observed. The solution is then neutralized with 1M HCl. About 200 uL of various concentrations of the degraded sample products are placed in 96-well tissue culture plates and seeded with human gastric carcinoma cells (GT3TKB) at 104/well density. The degraded sample products are incubated with the GT3TKB cells for 48 hours.

The results of the assay may be plotted as % relative growth vs. concentration of degraded sample in the tissue culture well. In addition, polymers, polymer matrices, and formulations of the present invention may also be evaluated by well-known in vivo tests, such as subcutaneous implantations in rats to confirm that they do not cause significant levels of irritation or inflammation at the subcutaneous implantation sites.

The term “biodegradable” is art-recognized, and includes polymers, polymer matrices, gels, compositions and formulations, such as those described herein, that are intended to degrade during use. Biodegradable polymers and matrices typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to side chain or that connects a side chain to the polymer backbone. For example, a therapeutic agent, biologically active agent, or other chemical moiety attached as a side chain to the polymer backbone may be released by biodegradation. In certain embodiments, one or the other or both generally types of biodegradation may occur during use of a polymer. As used herein, the term “biodegradation” encompasses both general types of biodegradation.

The degradation rate of a biodegradable polymer often depends in part on a variety of factors, including the chemical identity of the linkage responsible for any degradation, the molecular weight, crystallinity, biostability, and degree of cross-linking of such polymer, the physical characteristics of the implant, shape and size, and the mode and location of administration. For example, the greater the molecular weight, the higher the degree of crystallinity, and/or the greater the biostability, the biodegradation of any biodegradable polymer is usually slower. The term “biodegradable” is intended to cover materials and processes also termed “bioerodible”.

In certain embodiments, the biodegradation rate of such polymer may be characterized by the presence of enzymes, for example a chondroitinase. In such circumstances, the biodegradation rate may depend on not only the chemical identity and physical characteristics of the polymer matrix, but also on the identity of any such enzyme.

In certain embodiments, polymeric formulations of the present invention biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 6 and 8 having a temperature of between about 25° and 37° C. In other embodiments, the polymer degrades in a period of between about one hour and several weeks, depending on the desired application. In some embodiments, the polymer or polymer matrix may include a detectable agent that is released upon degradation.

The term “cartilage degradation activity” refers to an activity or the presence of a substance that may lead to the degradation of cartilage, for example, the activity or presence of degrading enzymes, or the presence of fibrillation, erosion or cracking on the cartilage.

The term “cartilage forming cells” include cells that form or promote formation of cartilage. Such cells include chondrocytes and mesenchymal stem cells.

The term “cross-linked” herein refers to a composition containing intermolecular cross-links and optionally intramolecular cross-links, arising from the formation of covalent bonds. Covalent bonding between two cross-linkable components may be direct, in which case an atom in one component is directly bound to an atom in the other component, or it may be indirect, through a linking group. A cross-linked gel or polymer matrix may, in addition to covalent bonds, also include intermolecular and/or intramolecular noncovalent bonds such as hydrogen bonds and electrostatic (ionic) bonds. The term “cross-linkable” refers to a component or compound that is capable of undergoing reaction to form a cross-linked composition.

“Electromagnetic radiation” as used in this specification includes, but is not limited to, radiation having the wavelength of 10-2 to 10 meters. Particular embodiments of electromagnetic radiation of the present invention employ the electromagnetic radiation of: gamma-radiation (10-2 to 1-13 m), x-ray radiation (10-11 to 10-9 m), ultraviolet light (10 nm to 400 nm), visible light (400 nm to 700 nm), infrared radiation (700 nm to 1.0 mm), and microwave radiation (1 mm to 30 cm).

The term “functionalized” refers to a modification of an existing molecular segment to generate or introduce a new reactive functional group (e.g., acrylate group) that is capable of undergoing reaction with another functional group (e.g., a sulfhydryl group) to form a covalent bond. For example, carboxylic acid groups can be functionalized by reaction with an acyl halide, e.g., an acyl chloride, again using known procedures, to provide a new reactive functional group in the form of an anhydride.

The term “gel” refers to a state of matter between liquid and solid, and is generally defined as a cross-linked polymer network swollen in a liquid medium. Typically, a gel is a two-phase colloidal dispersion containing both solid and liquid, wherein the amount of solid is greater than that in the two-phase colloidal dispersion referred to as a “sol.” As such, a “gel” has some of the properties of a liquid (i.e., the shape is resilient and deformable) and some of the properties of a solid (i.e., the shape is discrete enough to maintain three dimensions on a two dimensional surface.) “Gelation time” also referred to herein as “gel time,” refers to the time it takes for a composition to become non-flowable under modest stress. This is generally exhibited as reaching a physical state in which the elastic modulus G′ equals or exceeds the viscous modulus G″, i.e., when tan (delta) becomes 1 (as may be determined using conventional Theological techniques).

The term “hydrogel” is used to refer to water-swellable polymeric matrices that can absorb a substantial amount of water, for example, between 70% to 90% water, or more, to form elastic gels, wherein “matrices” are three-dimensional networks of macromolecules held together by covalent or noncovalent crosslinks. Upon placement in an aqueous environment, dry hydrogels swell to the extent allowed by the degree of cross-linking.

Hydrogels consist of hydrophilic polymers cross-linked to from a water-swollen, insoluble polymer network. Cross-linking can be initiated by many physical or chemical mechanisms. Photopolymerization is a method to covalently crosslink polymer chains, whereby a photoinitiator and polymer solution (termed “pre-gel” solution) are exposed to a light source specific to the photoinitiator. Upon activation, the photoinitiator reacts with specific functional groups in the polymer chains, crosslinking them to form the hydrogel. The reaction is rapid (3-5 minutes) and proceeds at room and body temperature. Photoinduced gelation enables spatial and temporal control of scaffold formation, permitting shape manipulation after injection and during gelation in vivo. Cells and bioactive factors can be easily incorporated into the hydrogel scaffold by simply mixing with the polymer solution prior to photogelation.

Photopolymerizable materials have been used in a wide variety of biomedical applications, including dentistry, drug delivery, and tissue engineering.

Hydrogels of interest are semi-interpenetrating networks that promote cartilage repair while discouraging scar formation. The hydrogels of interest are derivatized to be reactive with functional groups found on a primer of interest. Hydrogels of interest also are configured to have a viscosity that will enable the gelled hydrogel to remain affixed on or in the cartilage. Control of viscosity can be controlled by the monomers and polymers used, by the level of water trapped in the hydrogel and by incorporated thickeners, such as biopolymers, such as proteins, lipids, saccharides and the like. An example of such a thickener is hyaluronic acid.

A “polymerizing initiator” refers to any substance or stimulus that can initiate polymerization of monomers or macromers by free radical generation. Exemplary polymerizing initiators include electromagnetic radiation, heat, and chemical compounds.

As used herein, the term “saccharide”, refers to a mono-, di-, tri-, or higher order saccharide or oligosaccharide. Representative monosaccharides include glucose, mannose, galactose, glucosamine, mannosamine, galactosamine, fructose, glyceraldehyde, erythrose, threose, ribose, arabinose, xylose, lyxose, allose, altrose, gluose, idose, talose, psicose, sorbose, and tagatose. Exemplary disaccharides include maltose, lactose, sucrose, cellobiose, trehalose, isomaltose, gentiobiose, melibiose, laminaribiose, chitobiose, xylobiose, mannobiose, sophorose, and the like. Certain tri- and higher oligosaccharides include raffinose, maltotriose, isomaltotriose, maltotetraose, maltopentaose, mannotriose, manninotriose, etc. Exemplary polysaccharides include starch, sodium starch glycolate, alginic acid, cellulose, carboxymethylcellulose, hydroxyethylcellulose, hydropropylcellulose, hydroxypropylmethylcellulose, ethylcellulose, carageenan, chitosan, chondroitin sulfate, heparin, hyaluronic acid, and pectinic acid.

As used herein, a “saccharide unit” refers to a saccharide molecule having at least one pyranose or furanose ring. In some embodiments, at least one hydrogen atom may be removed from a hydroxyl group of a saccharide unit, as when the hydroxyl group has been esterified.

The term “detectable agent” includes those agents that may be used for diagnostic purposes. Examples of such diagnostic agents include imaging agents that are capable of generating a detectable image. Such imaging agents shall include dyes, radionuclides and compounds containing them (e.g., tritium, iodine-125, iodine-131, iodine-123, iodine-124, astatine-210, carbon-11, carbon-14, nitrogen-13, fluorine-18, Tc-99m, Re-186, Ga-68, Re-188, Y-90, Sm-153, Bi-212, Cu-67, Cu-64, and Cu-62), unpaired spin atoms and free radicals (e.g., Fe, lanthanides, and Gd), contrast agents (e.g., chelated (DTPA) manganese), and fluorescent or chemiluminescent agents.

The term “treating” or “treatment” is an art-recognized term that includes curing as well as ameliorating at least one symptom of any condition or disease. Treating includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder or condition, e.g., causing regression of the disease, disorder and/or condition. Further, treating the disease or condition includes ameliorating at least one symptom of the particular disease or condition, even if the underlying pathophysiology is not affected.

“Viscosity” is understood herein as it is recognized in the art to be the internal friction of a fluid or the resistance to flow exhibited by a fluid material when subjected to deformation. The degree of viscosity of the polymer can be adjusted by the molecular weight of the polymer, as well as by mixing different isomers of the polymer backbone; other methods for altering the physical characteristics of a specific polymer will be evident to practitioners of ordinary skill with no more than routine experimentation. The molecular weight of the polymer used in the composition of the invention can vary widely, depending on whether a rigid solid state (usually higher molecular weights) is desirable, or whether a fluid state (usually lower molecular weights) is desired.

The term “pharmaceutically acceptable salts” is art-recognized, and includes relatively non-toxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66: 1-19 (1977).

A “patient,” “subject,” or “host” to be treated by the subject method may mean either a human or non-human animal, such as primates, mammals, and vertebrates.

The terms “prophylactic” or “therapeutic” treatment are art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “synovial fluid” refers to the liquid produced by the synovial membranes of a joint. Synovial fluid may act as a lubricant.

The terms “incorporated”, “encapsulated”, and “entrapped” are art-recognized when used in reference to a therapeutic agent, dye, or other material and a polymeric composition, such as a composition of the present invention. In certain embodiments, these terms include incorporating, formulating or otherwise including such agent into a composition that allows for sustained release of such agent in the desired application. The terms may contemplate any manner by which a therapeutic agent or other material is incorporated into a polymer matrix, including for example, attached to a monomer of such polymer (by covalent or other binding interaction) and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to the incorporation of a therapeutic agent or other material and at least one other therapeutic agent or other material in a subject composition.

More specifically, the physical form in which any therapeutic agent or other material is encapsulated in polymers may vary with the particular embodiment. For example, a therapeutic agent or other material may be first encapsulated in a microsphere and then combined with the polymer in such a way that at least a portion of the microsphere structure is maintained. Alternatively, a therapeutic agent or other material may be sufficiently immiscible in the polymer of the invention that it is dispersed as small droplets, rather than being dissolved, in the polymer. Any form of encapsulation or incorporation is contemplated by the present invention, in so much as the sustained release of any encapsulated therapeutic agent or other material determines whether the form of encapsulation is sufficiently acceptable for any particular use.

A “wound closing device” includes devices and materials that may close or assist in closing a wound, such as for example, sutures, staples, sealants, and glues or adhesives.

The term “aliphatic” is an art-recognized term and includes linear, branched, and cyclic alkanes, alkenes, or alkynes. In certain embodiments, aliphatic groups in the present invention are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C1-C30 for straight chain, C3-C30 for branched chain), and alternatively, about 20 or fewer. Likewise, cycloalkyls have from about 3 to about 10 carbon atoms in their ring structure, and alternatively about 5, 6 or 7 carbons in the ring structure.

Moreover, the term “alkyl” (or “lower alkyl”) includes both “unsubstituted alkyls” and “substituted alkyls”, the latter of which refers to alkyl moieties having substituents replacing a hydrogen on one or more carbons of the hydrocarbon backbone. Such substituents may include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like. Exemplary substituted alkyls are described below. Cycloalkyls may be further substituted with alkyls, alkenyls, alkoxys, alkylthios, aminoalkyls, carbonyl-substituted alkyls, —CF3, —CN, and the like.

The term “aralkyl” is art-recognized, and includes alkyl groups substituted with an aryl group (e.g., an aromatic or heteroaromatic group).

The terms “alkenyl” and “alkynyl” are art-recognized, and include unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to ten carbons, alternatively from one to about six carbon atoms in its backbone structure. Likewise, “lower alkenyl” and “lower alkynyl” have similar chain lengths.

A “methacrylate” refers to a vinylic carboxylate, for example, a methacrylic acid in which the acidic hydrogen has been replaced. Representative methacrylic acids include acrylic, methacrylic, α-chloroacrylic, α-cyano acrylic, α-ethylacrylic, maleic, fumaric, itaconic, and half esters of the latter dicarboxylic acids.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “aryl” is art-recognized, and includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF3, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls.

The terms ortho, meta and para are art-recognized and apply to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles.

Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like.

The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The terms “polycyclyl” and “polycyclic group” are art-recognized, and include structures with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “carbocycle” is art recognized and includes an aromatic or non-aromatic ring in which each atom of the ring is carbon. The following art-recognized terms have the following meanings: “nitro” means —N02; the term “halogen” designates —F, —Cl, —Br or —I; the term “sulfhydryl” means —SH; the term “hydroxyl” means —OH; and the term “sulfonyl” means —SO2—.

The terms “amine” and “amino” are art-recognized and include both unsubstituted and substituted amines. The amines may be substituted to produce secondary and tertiary amines. Thus, the term “alkylamine” includes an amine group, as defined above, having a substituted or unsubstituted alkyl attached thereto, i.e., at least one alkyl group. The term “acylamino” is art-recognized and includes a amine substituted with an acyl group as defined herein.

The term “amido” is art-recognized as an amino-substituted carbonyl.

The term “alkylthio” is art-recognized and includes an alkyl group, as defined above, having a sulfur radical attached thereto. In certain embodiments, the “alkylthio” moiety is represented by one of —S-alkyl, —S-alkenyl or —S-alkynyl.

The terms “alkoxyl” or “alkoxy” are art-recognized and include an alkyl group, as defined above, having an oxygen radical attached thereto. Representative alkoxyl groups include methoxy, ethoxy, propyloxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl or —O—(CH2).

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

The definition of each expression, e.g. alkyl, m, n, etc., when it occurs more than once in any structure, is intended to be independent of its definition elsewhere in the same structure unless otherwise indicated expressly or by the context.

The term “selenoalkyl” is art-recognized and includes an alkyl group having a substituted seleno group attached thereto. Exemplary “selenoethers” which may be substituted on the alkyl are selected from one of —Se-alkyl, —Se-alkenyl, and —Se-alkynyl.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms are art-recognized and represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain monomeric subunits of the present invention may exist in particular geometric or stereoisomeric forms. In addition, polymers and other compositions of the present invention may also be optically active. The present invention contemplates all such compounds, including cis- and trans-isomers, R- and S-enantiomers, diastereomers, (d)-isomers, (l)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

For purposes of this invention, the chemical elements are identified in accordance with the Periodic Table of the Elements, CAS version, Handbook of Chemistry and Physics, 67th Ed., 1986-87, inside cover.

The term “hydrocarbon” is art recognized and includes all permissible compounds having at least one hydrogen and one carbon atom. For example, permissible hydrocarbons include acyclic and cyclic, branched and unbranched, carbocyclic and heterocyclic, aromatic and nonaromatic organic compounds that may be substituted or unsubstituted.

The phrase “protecting group” is art-recognized and includes temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed. Greene et al., Protective Groups in Organic Synthesis 2nd ed., Wiley, New York, (1991).

The phrase “hydroxyl-protecting group” is art-recognized and includes those groups intended to protect a hydroxyl group against undesirable reactions during synthetic procedures and includes, for example, benzyl or other suitable esters or ethers groups known in the art.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (o) constant. This well known constant is described in many references, for instance, March, Advanced Organic Chemistry 251-59, McGraw Hill Book Company, New York, (1977). The Hammett constant values are generally negative for electron donating groups (o (P)=−0.66 for NH2) and positive for electron withdrawing groups (cy (P)=0.78 for a nitro group), a (P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

In some embodiments, this disclosure is directed to a composition comprising at least one monomeric unit of a saccharide or other biocompatible monomer or polymer, wherein the monomers have reactive sites that will enable at least two functional groups or substituents, such as chondroitin sulfate, functionalized by at least two polymerizable moieties. Chondroitin sulfate is a natural component of cartilage and may be a useful scaffold material for its regeneration. Chondroitin sulfate includes members of 10-60 kDa glycosaminoglycans. The repeat units, or monomeric units, of chondroitin sulfate consist of a disaccharide, β(1-4)-linked D-glucuronyl β(1-3) N-acetyl-D-galactosamine sulfate.

A polymerizable moiety includes any moiety that is capable of polymerizing upon exposure to a polymerizing initiator. A polymerizable moiety may include alkenyl moieties such as acrylates, methacrylates, dimethacrylates, oligoacrylates, oligomethoacrylates, ethacrylates, itaconates and acrylamides, all of which can be functionalized or substituted as taught herein. Further polymerizable moieties include aldehydes.

Other polymerizable moities may include ethylenically unsaturated monomers including, for example, alkyl esters of acrylic or methacrylic acid such as methyl methacrylate, ethyl methacrylate, butyl methacrylate, ethyl acrylate, butyl acrylate, hexyl acrylate, n-octyl acrylate, lauryl methacrylate, 2-ethylhexyl methacrylate, nonyl acrylate, benzyl methacrylate, the hydroxyalkyl esters of the same acids such as 2-hydroxyethyl acrylate, 2-hydroxyethyl methacrylate, and 2-hydroxypropyl methacrylate, the nitrile and amides of the same acids such as acrylonitrile, methacrylonitrile, and methacrylamide, vinyl acetate, vinyl propionate, vinylidene chloride, vinyl chloride, and vinyl aromatic compounds such as styrene, t-butyl styrene and vinyl toluene, dialkyl maleates, dialkyl itaconates, dialkyl methylene-malonates, isoprene, and butadiene. Suitable ethylenically unsaturated monomers containing carboxylic acid groups include acrylic monomers such as acrylic acid, methacrylic acid, ethacrylic acid, itaconic acid, maleic acid, fumaric acid, monoalkyl itaconate including monomethyl itaconate, monoethyl itaconate, and monobutyl itaconate, monoalkyl maleate including monomethyl maleate, monoethyl maleate, and monobutyl maleate, citraconic acid, and styrene carboxylic acid. Suitable polyethylenically unsaturated monomers include butadiene, isoprene, allylmethacrylate, diacrylates of alkyl diols such as butanediol diacrylate and. hexanediol diacrylate, divinyl benzene and the like.

Cross-linked polymer matrices of the present invention may include hydrogels. The water content of a hydrogel may provide information on the pore structure. Further, the water content may be a factor that influences, for example, the survival of encapsulated cells within the hydrogel. The amount of water that a hydrogel is able to absorb may be related to the cross-linking density and/or pore size. For example, the percentage of methacrylate groups on a functionalized macromer, such as chondroitin sulfate or keratin sulfate, may dictate the amount of water absorbable.

The polymerizable agent of the present invention may comprise monomers, macromers, oligomers, polymers, or a mixture thereof. The polymer compositions can consist solely of covalently crosslinkable polymers, or ionically crosslinkable polymers, or polymers crosslinkable by redox chemistry, or polymers crosslinked by hydrogen bonding, or any combination thereof. The polymerizable agent should be substantially hydrophilic and biocompatible.

Suitable hydrophilic polymers include synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxylated celluloses such as hydroxyethyl cellulose and methylhydroxypropyl cellulose, and natural polymers such as polypeptides, polysaccharides or carbohydrates such as Ficoll™, polysucrose, hyaluronic acid, dextran, heparan sulfate, chondroitin sulfate, heparin, or alginate, and proteins such as gelatin, collagen, albumin, or ovalbumin or copolymers or blends thereof. As used herein, “celluloses” includes cellulose and derivatives of the types described above; “dextran” includes dextran and similar derivatives thereof.

Examples of materials that can be used to form a hydrogel include modified alginates. Alginate is a carbohydrate polymer isolated from seaweed, which can be crosslinked to form a hydrogel by exposure to a divalent cation such as calcium, as described, for example in WO 94/25080, the disclosure of which is incorporated herein by reference. Alginate is ionically crosslinked in the presence of divalent cations, in water, at room temperature, to form a hydrogel matrix. Modified alginate derivatives may be synthesized which have an improved ability to form hydrogels. The use of alginate as the starting material is advantageous because it is available from more than one source, and is available in good purity and characterization. As used herein, the term “modified alginates” refers to chemically modified alginates with modified hydrogel properties. Naturally occurring alginate may be chemically modified to produce alginate polymer derivatives that degrade more quickly. For example, alginate may be chemically cleaved to produce smaller blocks of gellable oligosaccharide blocks and a linear copolymer may be formed with another preselected moiety, e.g. lactic acid or epsilon-caprolactone. The resulting polymer includes alginate blocks that permit ionically catalyzed gelling, and oligoester blocks that produce more rapid degradation depending on the synthetic design. Alternatively, alginate polymers may be used wherein the ratio of mannuronic acid to guluronic acid does not produce a film gel, which arc derivatized with hydrophobic, water-labile chains, e.g., oligomers of epsilon-caprolactone. The hydrophobic interactions induce gelation, until they degrade in the body.

Alginate is ionically crosslinked in the presence of divalent cations, in water, at room temperature, to form a hydrogel matrix. Due to these mild conditions, alginate has been the most commonly used polymer for hybridoma cell encapsulation, as described, for example, in U.S. Pat. No. 4,352,883 to Lim. In the Lim process, an aqueous solution containing the biological materials to be encapsulated is suspended in a solution of a water soluble polymer, the suspension is formed into droplets which are configured into discrete microcapsules by contact with multivalent cations, then the surface of the microcapsules is crosslinked with polyamino acids to form a semipermeable membrane around the encapsulated materials.

Modified alginate derivatives may be synthesized which have an improved ability to form hydrogels. The use of alginate as the starting material is advantageous because it is available from more than one source, and is available in good purity and characterization. As used herein, the term “modified alginates” refers to chemically modified alginates with modified hydrogel properties. Naturally occurring alginate may be chemical modified to produce alginate polymer derivatives that degrade more quickly. For example, alginate may be chemically cleaved to produce smaller blocks of gellable oligosaccharide blocks and a linear copolymer may be formed with another preselected moiety, e.g. lactic acid or ε-caprolactone. The resulting polymer includes alginate blocks that permit ionically catalyzed gelling, and oligoester blocks that produce more rapid degradation depending on the synthetic design. Alternatively, alginate polymers may be used, wherein the ratio of mannuronic acid to guluronic acid does not produce a firm gel, which are derivatized with hydrophobic, water-labile chains, e.g., oligomers of ε-caprolactone. The hydrophobic interactions induce gelation, until they degrade in the body.

Additionally, polysaccharides which gel by exposure to monovalent cations, including bacterial polysaccharides, such as gellan gum, and plant polysaccharides, such as carrageenans, may be crosslinked to form a hydrogel using methods analogous to those available for the crosslinking of alginates described above. Polysaccharides that gel in the presence of monovalent cations form hydrogels upon exposure, for example, to a solution comprising physiological levels of sodium. Hydrogel precursor solutions also may be osmotically adjusted with a nonion, such as mannitol, and then injected to form a gel.

Polysaccharides that are very viscous liquids or are thixotropic, and form a gel over time by the slow evolution of structure, are also useful. For example, hyaluronic acid, which forms an injectable gel with a consistency like a hair gel, may be utilized. Modified hyaluronic acid derivatives are particularly useful. As used herein, the term “modified hyaluronic acids” refers to chemically modified hyaluronic acids. Modified hyaluronic acids may be designed and synthesized with preselected chemical modifications to adjust the rate and degree of crosslinking and biodegradation. For example, modified hyaluronic acids may be designed and synthesized which are esterified with a relatively hydrophobic group such as propionic acid or benzylic acid to render the polymer more hydrophobic and gel-forming, or which are grafted with amines to promote electrostatic self-assembly. Modified hyaluronic acids thus may be synthesized which are injectable, in that they flow under stress, but maintain a gel-like structure when not under stress. Hyaluronic acid and hyaluronic derivatives are available from Genzyme, Cambridge, Mass. and Fidia, Italy.

Other polymeric hydrogel precursors include polyethylene oxide-polypropylene glycol block copolymers such as Pluronics™ or Tetronics™, which are crosslinked by hydrogen bonding and/or by a temperature change, as described in Steinleitner et al., Obstetrics & Gynecology, 77:48-52 (1991); and Steinleitner et al., Fertility and Sterility, 57:305-308 (1992). Other materials that may be utilized include proteins such as fibrin, collagen and gelatin. Polymer mixtures also may be utilized. For example, a mixture of polyethylene oxide and polyacrylic acid that gels by hydrogen bonding upon mixing may be utilized. In one embodiment, a mixture of a 5% w/w solution of polyacrylic acid with a 5% w/w polyethylene oxide (polyethylene glycol, polyoxyethylene) 100,000 can be combined to form a gel over the course of time, e.g., as quickly as within a few seconds.

Covalently crosslinkable hydrogel precursors also are useful. For example, a water soluble polyamine, such as chitosan, can be cross-linked with a water soluble diisothiocyanate, such as polyethylene glycol diisothiocyanate. The isothiocyanates will react with the amines to form a chemically crosslinked gel. Aldehyde reactions with amines, e.g., with polyethylene glycol dialdehyde also may be utilized. A hydroxylated water soluble polymer also may be utilized.

Alternatively, polymers may be utilized which include substituents that are crosslinked by a radical reaction upon contact with a radical initiator. For example, polymers including ethylenically unsaturated groups that can be photochemically crosslinked may be utilized, as disclosed in WO 93/17669, the disclosure of which is incorporated herein by reference. In this embodiment, water soluble macromers that include at least one water soluble region, a biodegradable region, and at least two free radical-polymerizable regions, are provided. The macromers are polymerized by exposure of the polymerizable regions to free radicals generated, for example, by photosensitive chemicals and or light. Examples of these macromers are PEG-oligolactyl-acrylates, wherein the acrylate groups are polymerized using radical initiating systems, such as an eosin dye, or by brief exposure to ultraviolet or visible light. Additionally, water soluble polymers, which include cinnamoyl groups that may be photochemically crosslinked, may be utilized, as disclosed in Matsuda et al., ASAID Trans., 38:154-157 (1992).

In general, the polymers are at least partially soluble in aqueous solutions, such as water, buffered salt solutions, or aqueous alcohol solutions. Methods for the synthesis of the other polymers described above are known to those skilled in the art. See, for example Concise Encyclopedia of Polymer Science and Polymeric Amines and Ammonium Salts, E. Goethals, editor (Pergamen Press, Elmsford, N.Y. 1980). Many polymers, such as poly(acrylic acid), are commercially available. Naturally occurring and synthetic polymers may be modified using chemical reactions available in the art and described, for example, in March, “Advanced Organic Chemistry,” 4th Edition, 1992, Wiley-Interscience Publication, New York.

Water soluble polymers with charged side groups may be crosslinked by reacting the polymer with an aqueous solution containing ions of the opposite charge, either cations if the polymer has acidic side groups or anions if the polymer has basic side groups. Examples of cations for crosslinking of the polymers with acidic side groups to form a hydrogel are monovalent cations such as sodium, and multivalent cations such as copper, calcium, aluminum, magnesium, strontium, barium, and tin, and di-, tri- or tetra-functional organic cations such as alkylammonium salts. Aqueous solutions of the salts of these cations are added to the polymers to form soft, highly swollen hydrogels and membranes. The higher the concentration of cation, or the higher the valence, the greater the degree of cross-linking of the polymer. Additionally, the polymers may be crosslinked enzymatically, e.g., fibrin with thrombin.

In the embodiment wherein modified alginates and other anionic polymers that can form hydrogels which are malleable are used to encapsulate cells, the hydrogel is produced by cross-linking the polymer with the appropriate cation, and the strength of the hydrogel bonding increases with either increasing concentrations of cations or of polymer. Concentrations from as low as 0.001 M have been shown to cross-link alginate. Higher concentrations are limited by the toxicity of the salt.

The preferred anions for cross-linking of the polymers to form a hydrogel are monovalent, divalent or trivalent anions such as low molecular weight dicarboxylic acids, for example, terepthalic acid, sulfate ions and carbonate ions. Aqueous solutions of the salts of these anions are added to the polymers to form soft, highly swollen hydrogels and membranes, as described with respect to cations.

A variety of polycations can be used to complex and thereby stabilize the polymer hydrogel into a semi-permeable surface membrane. Examples of materials that can be used include polymers having basic reactive groups such as amine or imine groups, having a preferred molecular weight between 3,000 and 100,000, such as polyethylenimine and polylysine. These are commercially available. One polycation is poly(L-lysine); examples of synthetic polyamines are: polyethyleneimine, poly(vinylamine), and poly(allyl amine). There are also natural polycations such as the polysaccharide, chitosan.

Suitable ionically crosslinkable groups include phenols, amines, imines, amides, carboxylic acids, sulfonic acids and phosphate groups. Negatively charged groups, such as carboxylate, sulfonate and phosphate ions, can be crosslinked with cations such as calcium ions. The crosslinking of alginate with calcium ions is an example of this type of ionic crosslinking. Positively charged groups, such as ammonium ions, can be crosslinked with negatively charged ions such as carboxylate, sulfonate and phosphate ions. Preferably, the negatively charged ions contain more than one carboxylate, sulfonate or phosphate group.

The preferred anions for cross-linking of the polymers to form a hydrogel are monovalent, divalent or trivalent anions such as low molecular weight dicarboxylic acids, for example, terepthalic acid, sulfate ions and carbonate ions. Aqueous solutions of the salts of these anions are added to the polymers to form soft, highly swollen hydrogels and membranes, as described with respect to cations.

Polyanions that can be used to form a semi-permeable membrane by reaction with basic surface groups on the polymer hydrogel include polymers and copolymers of acrylic acid, methacrylic acid, and other derivatives of acrylic acid, polymers with pendant SO3H groups such as sulfonated polystyrene, and polystyrene with carboxylic acid groups. These polymers can be modified to contain active species polymerizable groups and/or ionically crosslinkable groups. Methods for modifying hydrophilic polymers to include these groups are well known to those of skill in the art.

The polymers may be intrinsically biodegradable, but are preferably of low biodegradability (for predictability of dissolution) but of sufficiently low molecular weight to allow excretion. The maximum molecular weight to allow excretion in human beings (or other species in which use is intended) will vary with polymer type, but will often be about 20,000 daltons or below. Usable, but less preferable for general use because of intrinsic biodegradability, are water-soluble natural polymers and synthetic equivalents or derivatives, including polypeptides, polynucleotides, and degradable polysaccharides.

The polymers can be a single block with a molecular weight of at least 600, preferably 2000 or more, and more preferably at least 3000. Alternatively, the polymers can include can be two or more water-soluble blocks which are joined by other groups. Such joining groups can include biodegradable linkages, polymerizable linkages, or both. For example, an unsaturated dicarboxylic acid, such as maleic, fumaric, or aconitic acid, can be esterified with hydrophilic polymers containing hydroxy groups, such as polyethylene glycols, or amidated with hydrophilic polymers containing amine groups, such as poloxamines. For example, see U.S. 2004/0170663.

For example, PEODA may be used in a polymer system for cartilage tissue engineering, and cross-linked polymer matrices may include cogels of CS-MA and PEODA The COMA hydrogels may absorb more water than the PEODA hydrogels, thus, increasing the percentage of CS-MA in the cogels increases the water content.

The mechanical properties of a cross-linked polymer matrix, such as a hydrogel scaffold may also be related to the hydrogel pore structure. For applications in tissue engineering, scaffolds with different mechanical properties may be desirable depending on the desired clinical application. For example, scaffolds for cartilage tissue engineering in the articular joint must survive higher mechanical stresses than a cartilage tissue engineering system implanted subcutaneously for plastic surgery applications. Thus, hydrogels with mechanical properties that are easily manipulated may be desired.

The dynamic frequency-sweep experiments disclosed herein show that hydrogels with various PEODA/CS-MA ratios were elasticity dominant and not sensitive to the shear frequency. The norm of the dynamic shear modulus G*j increases with the shear frequency, however, such increase may be insignificant compared with the average value of 1G*1. The phase angle 8 is narrowly ranged between about 1 and about 6 for all frequencies and all weight ratios. This may indicate that the Theological properties of PEODA and CS-MA are similar and the copolymerization does not alter these properties significantly. Cogels with higher portion of PEODA (100% and 75%) have a higher mechanical strength (indicated by 1G*1) while the cogels with 50%, 25% and 0% PEODA exhibited a decrease of 1G*1 with the PEODA concentration. The 100% and 75% samples had a G* value 3-4 times that of the CS-MA gel. This is consistent with the swelling experiments that demonstrated that the PEODA gels are more highly cross-linked than the CS-MA gel.

Morphological analysis of the gels confirmed the CS-MA and PEODA hydrogel pore structure suggested by the swelling and mechanical analysis. As suggested by the swelling and mechanical data, the CS-MA gels exhibited a larger pore structure compared to the PEODA gels both on the surface and in the interior. SEM morphological studies demonstrated a uniform pore structure, both on the surface and in the interior of the gels. The reproducibility (low standard deviation) of the swelling and mechanical data also suggests that chondroitin sulfate is substituted and forms hydrogels in a uniform and consistent manner.

Hydrogels of interest can contain one or more pharmaceutically active agents, such as hormones, antibiotics, growth factors and so on.

The general criteria for pre-polymers (referred to herein also as macromers) that can be polymerized in contact with biological materials or cells are that: they are water-soluble or substantially water soluble, they can be further polymerized or crosslinked by free radical polymerization, they are non-toxic and they are too large to diffuse into cells, i.e., greater than 200 molecular weight Substantially water soluble is defined herein as being soluble in a mixture of water and organic solvent(s), where water makes up the majority of the mixture of solvents.

As used herein, the macromers must be photopolymerizable with light alone or in the presence of an initiator and/or catalyst, such as a free radical photoinitiator, wherein the light is in the visible or long wavelength ultraviolet range, that is, greater than or equal to 320 nm. Other reactive conditions may be suitable to initiate free radical polymerization if they do not adversely affect the viability of the living tissue to be encapsulated. The macromers must also not generate products or heat levels that are toxic to living tissue during polymerization. The catalyst or free radical initiator must also not be toxic under the conditions of use.

Examples of suitable polymers include polyethylene glycol (PEG) diacrylate, from a PEG diol; PEG triacrylate, formed from a PEG triol; PEG-cyclodextrin tetraacrylate, formed by grafting PEG to a cyclodextrin central ring, and further acrylating; PEG tetraacrylate, formed by grafting two PEG diols to a bis epoxide and further acrylating; hyaluronic acid methacrylate, formed by acrylating many sites on a hyaluronic acid chain; PEG-hyaluronic acid multiacrylate, formed by grafting PEG to hyaluronic acid and further acrylating; and PEG-unsaturated diacid ester formed by esterifying a PEG diol with an unsaturated diacid.

Polysaccharides include, for example, alginate, hyaluronic acid, chondroitin sulfate, dextran, dextran sulfate, heparin, heparin sulfate, heparan sulfate, chitosan, gellan gum, xanthan gum, guar gum, and K-carrageenan. Proteins, for example, include gelatin, collagen, elastin and albumin, whether produced from natural or recombinant sources.

Photopolymerizable substituents preferably include acrylates, diacrylates, oligoacrylates, dimethacrylates, or oligomethoacrylates, and other biologically acceptable photopolymerizable groups.

The water-soluble macromer may be derived from water-soluble polymers including, but not limited to, poly(ethylene oxide) (PEO), PEG, poly(vinyl alcohol) (PVA), poly(vinylpyrrolidone) (PVP), poly(ethyloxazoline) (PEOX) polyaminoacids, pseudopolyamino acids, and polyethyloxazoline, as well as copolymers of these with each other or other water soluble polymers or water insoluble polymers, provided that the conjugate is water soluble. An example of a water soluble conjugate is a block copolymer of polyethylene glycol and polypropylene oxide, commercially available as a Pluronic™ surfactant.

Polysaccharides such as alginate, hyaluronic acid, chondroitin sulfate, dextran, dextran sulfate, heparin, heparin sulfate, heparan sulfate, chitosan, gellan gum, xanthan gum, guar gum, water soluble cellulose derivatives, and carrageenan, which are linked by reaction with hydroxyls or amines on the polysaccharides can also be used to form the macromer solution.

Proteins such as gelatin, collagen, elastin, zein, and albumin, whether produced from natural or recombinant sources, which are made free-radical polymerization by the addition of carbon-carbon double or triple bond-containing moieties, including acrylate, diacrylate, methacrylate, ethacrylate, 2-phenyl acrylate, 2-chloro acrylate, 2-bromo acrylate, itaconate, oliogoacrylate, dimethacrylate, oligomethacrylate, acrylamide, methacrylamide, styrene groups, and other biologically acceptable photopolymerizable groups, can also be used to form the macromer solution.

Dye-sensitized polymerization is well known in the chemical literature. For example, light from an argon ion laser (514 nm), in the presence of an xanthin dye and an electron donor, such as triethanolamine, to catalyze initiation, serves to induce a free radical polymerization of the acrylic groups in a reaction mixture (Neckers, et al., (1989) Polym. Materials Sci. Eng., 60:15; Fouassier, et al., (1991) Makromol. Chem., 192:245-260). After absorbing the laser light, the dye is excited to a triplet state. The triplet state reacts with a tertiary amine such as the triethanolamine, producing a free radical that initiates the polymerization reaction. Polymerization is extremely rapid and is dependent on the functionality of the macromer and its concentration, light intensity, and the concentration of dye and amine.

Any dye can be used which absorbs light having a frequency between 320 nm and 900 nm, can form free radicals, is at least partially water soluble, and is non-toxic to the biological material at the concentration used for polymerization. There are a large number of photosensitive dyes that can be used to optically initiate polymerization, such as ethyl eosin, eosin Y, fluorescein, 2,2-dimethoxy-2-phenyl acetophenone, 2-methoxy, 2-phenylacetophenone, camphorquinone, rose bengal, methylene blue, erythrosin, phloxime, thionine, riboflavin, methylene green, acridine orange, xanthine dye, and thioxanthine dyes.

The preferred initiator dye is ethyl eosin due to its spectral properties in aqueous solution (absorption max=528 nm, extinction coefficient-1.1×105M-1cm-1, fluorescence max=547 nm, quantum yield=0.59). The dye bleaches after illumination and reaction with amine into a colorless product, allowing further beam penetration into the reaction system.

The catalysts useful with the photoinitiating dyes are nitrogen based compounds capable of stimulating the free radical reaction. Primary, secondary, tertiary or quaternary amines are suitable cocatalysts, as are any nitrogen atom containing electron-rich molecules. Cocatalysts include, but are not limited to, triethanolamine, triethylamine, ethanolamine, N-methyl diethanolamine, N,N-dimethyl benzylamine, dibenzyl amine, N-benzyl ethanolamine, N-isopropyl benzylamine, tetramethyl ethylenediamine, potassium persulfate, tetramethyl ethylenediamine, lysine, omithine, histidine and arginine.

Examples of the dye/photoinitiator system includes ethyl eosin with an amine, eosin Y with an amine, 2,2-dimethoxy-2-phenoxyacetophenone, 2-methoxy-2-phenoxyacetophenone, camphorquinone with an amine, and rose bengal with an amine.

In some cases, the dye may absorb light and initiate polymerization, without any additional initiator such as the amine. In these cases, only the dye and the macromer need be present to initiate polymerization upon exposure to light. The generation of free radicals is terminated when the laser light is removed. Some photoinitiators, such as 2,2-dimethoxy-2-phenylacetophenone, do not require any auxiliary amine to induce photopolymerization; in these cases, only the presence of dye, macromer, and appropriate wavelength light is required.

Preferred light sources include various lamps and lasers such as those described in the following examples, which have a wavelength of about 320-800 nm, most preferably about 365 nm or 514 nm.

This light can be provided by any appropriate source able to generate the desired radiation, such as a mercury lamp, long wave UV lamp, He—Ne laser, or an argon ion laser, or through the use of fiber optics.

Means other than light can be used for polymerization. Examples include initiation by thermal initiators, which form free radicals at moderate temperatures, such as benzoyl peroxide, with or without triethanolamine, potassium persulfate, with or without tetramethylethylenediamine, and ammonium persulfate with sodium bisulfite.

The water soluble macromers can be polymerized around biologically active molecules to form a delivery system for the molecules or polymerized around cells, tissues, sub-cellular organelles or other sub-cellular components to encapsulate the biological material. The water soluble macromers can also be polymerized to incorporate biologically active molecules to impart additional properties to the polymer, such as resistance to bacterial growth or decrease in inflammatory response, as well as to encapsulate tissues. A wide variety of biologically active material can be encapsulated or incorporated, including proteins, peptides, polysaccharides, organic or inorganic drugs, nucleic acids, sugars, cells, and tissues.

Examples of cells that can be encapsulated include primary cultures as well as established cell lines, including transformed cells. These include but are not limited to pancreatic islet cells, human foreskin fibroblasts, Chinese hamster ovary cells, beta cell insulomas, lymphoblastic leukemia cells, mouse 3T3 fibroblasts, dopamine secreting ventral mesencephanol cells, neuroblastoid cells, adrenal medulla cells, and T-cells. As can be seen from this partial list, cells of all types, including dermal, neural, blood, organ, muscle, glandular, reproductive, and immune system cells, as well as species of origin, can be encapsulated successfully by this method. Examples of proteins which can be encapsulated include hemoglobin, enzymes such as adenosine deaminase, enzyme systems, blood clotting factors, inhibitors or clot dissolving agents such as streptokinase and tissue plasminogen activator, antigens for immunization, and hormones, polysaccharides such as heparin, oligonucleotides such as antisense, bacteria and other microbial organisms, including viruses, vitamins, cofactors, and retroviruses for gene therapy can be encapsulated by these techniques.

The biological material can be first enclosed in a structure such as a polysaccharide gel. (Lim, U.S. Pat. No. 4,352,883; Lim, U.S. Pat. No. 4,391,909; Lim, U.S. Pat. No. 4,409,331; Tsang, et al., U.S. Pat. No. 4,663,286; Goosen et al., U.S. Pat. No. 4,673,556; Goosen et al., U.S. Pat. No. 4,689,293; Goosen et al., U.S. Pat. No. 4,806,355; Rha et al., U.S. Pat. No. 4,744,933; Rha et al., U.S. Pat. No. 4,749,620, incorporated herein by reference.) Such gels can provide additional structural protection to the material, as well as a secondary level of perm-selectivity.

The hydrogels of interest can be made with monomers or polymers that contain reactive groups that facilitate the gelling process. Thus, the monomers or polymers contain functional groups and substituents that enable such reaction. For example, poly(ethylene oxide diacrylate) (PEODA) or poly(etheylene glycol diacrylate) (PEGDA) is a suitable polymer for making a hydrogel.

Alternatively, the hydrogel can contain a separate polymerizing reagent. Examples are as known in the art.

The liquid hydrogel reagent then is gelled using facilitators, initiators or catalysts as known in the art and suitable for the reagents used. For example, in the case of PEODA, exposure to light will begin the gelation reaction.

A polymerization reaction of the present invention can be conducted by conventional methods such as mass polymerization, solution (or homogeneous) polymerization, suspension polymerization, emulsion polymerization, radiation polymerization (using y-ray, electron beam or the like), or the like.

Polymerizing initiators include electromechanical radiation. Initiation of polymerization may be accomplished by irradiation with light at a wavelength of between about 200 to about 700 nm, or above about 320 nm or higher, or even between about 514 nm and about 365 nm. In some embodiments, the light intensity is about 10 mW/cm3.

Examples of other initiators are organic solvent-soluble initiators such as benzoyl peroxide, azobisisobutyronitrile (AIBN), di-tertiary butyl peroxide and the like, water soluble initiators such as ammonium persulfate (APS), potassium persulfate, sodium persulfate, sodium thiosulfate and the like, redox-type initiators which are combinations of such initiator and tetramethylethylene, Fe salt, sodium hydrogen sulfite or like reducing agent, etc.

Useful photoinitiators are those which can be used to initiate by free radical generation polymerization of monomers with minimal cytotoxicity. In some embodiments, the initiators may work in a short time frame, for example, minutes or seconds. Exemplary dyes for UV or visible light initiation include ethyl eosin 2,2-dimethoxy-2-phenyl acetophenone, 2-methoxy-2-phenylacetophenone, other acetophenone derivatives, and camphorquinone. In all cases, crosslinking and polymerization are initiated among macromers by a light-activated free-radical polymerization initiator such as 2,2-dimethoxy-2-phenylacetophenone or a combination of ethyl eosin (10-4 to 10-2 M) and triethanol amine (0.001 to 0.1 M), for example.

Other photooxidizable and photoreducible dyes that may be used to initiate polymerization include acridine dyes, for example, acriblarine; thiazine dyes, for example, thionine; xanthine dyes; for example, rose bengal; and phenazine dyes, for example, methylene blue. These may be used with cocatalysts such as amines, for example, triethanolamine ; sulphur compounds; heterocycles, for example, imidazole; enolates; organometallics; and other compounds, such as N-phenyl glycine.

Other initiators include camphorquinones and acetophenone derivatives.

Thermal polymerization initiator systems may also be used. Such systems that are unstable at 37° C. and would initiate free radical polymerization at physiological temperatures include, for example, potassium persulfate, with or without tetraamethyl ethylenediamine; benzoylperoxide, with or without triethanolamine; and ammonium persulfate with sodium bisulfite.

A suitable hydrogel formulation amenable to photogellation would be a solution of PEODA (Nektar, San Carlos, Calif.) with an amount of PEODA ranging from 1-15% w/v depending on the viscosity and hardness desired of the final hydrogel with a suitable amount of a photoinitiator, such as Irgacure 2959 (Ciba) in an amount of about 0.05% w/v, as recommended by the manufacturer. The amount of monomer can be present in a range of 1-14%, 1-13%; 2-12%, 3-11%, 4-10%, 5-10%, 6-9%, 7% or 8% w/v. Optionally, the mixture can contain a thickener, and the mixture can contain hyaluronic acid (Ufecore). A suitable amount is a design choice based on the desired firmness of the hydrogel, but can range from 1-10 mg/ml. The thickener can be present at 2, 3, 4, 5, 6, 7, 8, 9, 11, 12, 13, 14 or 15 mg/ml or more as desired by the artisan. The reagents are suspended in a suitable liquid vehicle, preferably a physiological pharmaceutically acceptable medium, such as buffered saline.

The instant invention also relates to a material that enhances integration of biomaterials to cartilage that are compatible with a minimally invasive approach. A derivatized biologically compatible polymer, and preferably a derivitized biopolymer, known for the purposes of the instant invention as a primer, is used in the process. For example, a suitable backbone polymer is chondroitin sulphate (CS) or keratin sulphate, both natural cartilage extracellular matrix molecules. However, other carbohydrates can be used as well.

The primer preferably is functionalized with two different functional groups. The functionalized primer contains as a first functional group, a group that is reactive with cartilage. Cartilage contains, for example, collagen, elastic fibers, proteoglycans, glycosaminoglycans, fibrin, hyaluronic acid and so on. Those components comprise proteins and polysaccharides, which contain, for example, reactive amino groups, hydroxyl groups, carboxyl groups, sulfhydryl groups, keto groups and so on, as known in the art. Thus the first functional group is one that is reactive with those reactive groups of proteins and polysaccharides. One example would be an aldehyde groups (ALD).

The second functional group is one that is suitable to act as a binding partner or linking partner with the hydrogel of interest. The second functional group is one that is not reactive or less reactive that the first functional group with proteins and polysaccharides. One example is an alkenyl group, such as, a methacrylate group (MA).

The result is a directional primer with one aspect reactive with cartilage and a second aspect reactive with the hydrogel. The primer thus adheres to the cartilage and serves as a point of adherence for the hydrogel. The primer thus acts to prime the cartilage tissue surface before the hydrogel is injected into the defect.

A suitable primer is one containing chondroitin sulfate derivatized with aldehyde groups and methacrylate groups. A solution of same, again prepared in a suitable pharmaceutically acceptable liquid medium, such as buffered saline, contains the difunctionalized chondroitin sulfate in a concentration from 1-50% w/v. Other mixtures may contain, 5, 10, 15, 20, 25, 30, 35, 40 or 45% w/v of primer compound. The actual amount of primer compound used is selectable by the artisan.

Thus, in the example of above, the aldehyde groups react with existing proteins on the cartilage surface. Once this reaction is complete (˜4 min), the pre-gel solution of hydrogel is placed in the defect. In the case of PEODA or PEGDA as reagent, on light exposure (6-8 mW/cm2, 365 nm UV light), the methacrylate groups in the primer will react with the same functional groups in the pre-gel solution and on the primer, resulting in a hydrogel covalently bonded to the cartilage matrix.

Methods for making the primers of interest are as known in the art, and taught, for example, in WO2004/029137.

To contain injected material in place at any site in the body, or to form a mold for the gelling hydrogel a film or patch can be used. Use of patches to contain the injectable liquid, ungelled hydrogel, such as PEODA, throughout the photopolymerization process allows molding of the gelling material. Therefore, a patch enables injection of liquid PEODA in any location within any joints practically and does not interfere with the photopolymerization process.

The instant invention also relates to a universal guide which is designed for accuracy and for modularity, allowing the surgeon to aim the center of the cartilage defect that is to be treated with arthroscopic hydrogel injection and which enables drilling of the local bone, microfracture, by approaching from the subchondral bone side. The system is easy to use with ergonomic design features and the reproducibility and accuracy gained from rigidity of design and secure locking of moving components. The design has certain features that are modifications of currently available arthroscopic cruciate ligament reconstruction guides. The universal guide has been adapted to accommodate the needs for the described surgical technique that mainly involves subchondral drilling and injection of hydrogel through the film layer to the defect. A unique feature presented is a modification of the current cannulated drill ends that is obtained by adding holes to the trephines. This allows cells to be drained to the target under the guidance of the drill. All these features can be combined with computer assisted navigation technologies to increase the accuracy and precision of subchondral bone marrow stimulation without disrupting the plate. This can be achieved by an antegrade drilling either by using the microfracture technique or drilling through the defect.

A goat study demonstrated that the clinical procedure is readily applied in a large animal joint in both an open surgical environment and early cartilage repair. Goats have a joint thickness to diameter ratio that closely mimics the human joint. Cartilage defects were induced in the femoral condyle and tibial plane of goats and the defects filled as described herein. The defects were or were nearly completely healed.

Subchondral bone marrow stimulation techniques mobilize blood/bone marrow into the defect or target tissue that enables differentiation of same into cartilaginous repair tissue. Once disruption of the vascularized cancellous bone has been performed, a fibrin clot is formed and to serve as a bed for pluripotent cells. Those cells eventually differentiate into “chondrocyte-like” (Allen A A, Fealy S, Panariello R, et al. Chondral injuries. Sports Med and Arthroscopy Review. 4:51-58, 1996.), cells that secrete type I, II and other collagen types inherent to native cartilage content as well as cartilage specific proteoglycans when the proper mechanical and biological cues are provided. The cells produce a fibroblastic repair tissue that on appearance and initial biopsy can have a hyaline-like quality. (Minas T, Nehrer S. Current concepts in the treatment of articular cartilage defects. Orthopaedics. 20:525-538, 1997. Friend T. Making high tech human repairs. USA Today. Sec. 6D:1, Aug. 12, 1997. Ratcliffe A, Mow V C. The structure, function, and biologic repair of articular cartilage. pp. 123-154. In: Friedlaender G E, Goldberg V M (ed): Bone and Cartilage Allografts. American Academy of Orthopaedic Surgeons, Park Ridge, Ill., 1991.)

Microfracture technique has been developed to enhance chondral resurfacing by providing a suitable environment for new tissue formation and taking advantage of the body's own healing potential. Specially designed awls are used to make multiple perforations, or microfractures, into the subchondral bone plate. Perforations are made as close together as possible, usually approximately 3 to 4 mm apart to avoid the subchondral bone plate fracture. The released marrow elements (including mesenchymal stem cells, growth factors, and other healing proteins) form a surgically induced super clot that provides an enriched environment for new tissue formation. However, the surgeon does not have a control on the release of growth factors into the area. Therefore, the technique relies on body's own healing potential and the rehabilitation program that is crucial to optimize the results of the surgery. It is hoped that ideal physical environment especially the mechanical stimulus (Darling E M, Athanasiou K A. Biomechanical strategies for articular cartilage regeneration. Ann Biomed Eng. October 2003;31(9):1114-24. Review. Hunter C J, Mouw J K, Levenston M E. Dynamic compression of chondrocyte-seeded fibrin gels: effects on matrix accumulation and mechanical stiffness. Osteoarthritis Cartilage. February 2004;12(2):117-30.) for the marrow mesenchymal stem cells to differentiate into articular cartilage-like cells is promoted, which is ultimately leading to development of a durable repair cartilage that fills the original defect. (Steadman J R, Rodkey W G, Rodrigo J J. Microfracture: surgical technique and rehabilitation to treat chondral defects. Clin Orthop. October 2001;(391 Suppl):S362-9. Review) Unfortunately, over time the histological characteristics change into more predominantly fibrocartilaginous tissue. This is most probably due to the early initiation of mechanical loading that effects the biomechanically unstable and less organized and integrated new tissue formation within the defect. Buckwalter J A, Mankin H J. Articular cartilage: part II-degeneration and osteoarthrosis, repair, regeneration and transplantation. J Bone Joint Surg Am. 79A:612-632, 1997.

Subchondral drilling consists of drilling through the defect to penetrate the subchondral bone. The technique was first popularized in the late 1950's by Pridie, (Pridie K H. A method of resurfacing osteoarthritic knee joints. J Bone Joint Surg Br. 41B:618, 1959.) and subsequent findings suggest the repair tissue introduced into the area can look like grossly like hyaline cartilage but histologically resembles fibrocartilage. (Shapiro F, Koide S, Glimcher M J: Cell origin and differentiation in the repair of full thickness defects of articular cartilage. J Bone Joint Surg. 75A:532-553, 1993.) Drilling through the articular surface has been criticized because of the possibility of cell death through heat necrosis and this might interfere with regeneration efforts and integration of cartilage with the defect surface.

Microfracture is another such technique in which the lesion is exposed, debrided, and a series of small fractures about 3 to 4 mm in depth are produced with an awl. Adjacent cartilage is debrided to a stable cartilaginous rim, and any loose fragments and fibrous tissue are removed. Popularized early by Steadman, (Kim H K, Moran M E, Salter R B. The potential for regeneration of articular cartilage in defects created by chondral shaving and subchondral abrasions. J Bone Joint Surg Am. 73:1301-15, 1991. Rodrigo J J, Steadman J R, Silliman J F. Osteoarticular injuries of the knee. pp. 2077-82. In: Chapman M W (ed): Operative Orthopaedics,. Vol. 3, 2nd Ed. Lippincott, Philadelphia, Pa., 1993.) microfracture has a few advantages over drilling: no heat necrosis, the awl creates more exposed surface area for clot formation, and the structural integrity of the subchondral bone is maintained. Although this method has been widely used in orthopedics, the formation of fibrocartilage could not be prevented.

Stimulating articular cartilage growth through the use of various grafting techniques has recently been reported. Utilizing autologous tissue or allografts, these procedures are designed to provide a suitable environment for stimulation of the mesenchymal cells to produce type II collagen fibers. The success of such approaches is at least partly related to the severity of the abnormalities, graft and technique utilized, age of the patient, joints involved, correction of associated pathology, weight bearing restrictions and the use of postoperative continuous passive motion. Wirth C J, Rudert M. Techniques of cartilage growth enhancement: a review of the literature. Arthroscopy: The Journal of Arthroscopic and Related Surgery. 12:300-308, 1996. Intact full thickness grafts suffer the problems of mismatched sizes, immunologic rejection, and tissue structural weakening during the process of revascularization.

Mesenchymal stem cells are currently procured from periosteum and bone marrow. The procurement of stem cells from these sources is tedious. Therefore, other sources of cells have been investigated, such as cells obtained from adipose tissue other than from bone marrow or periosteum. This method seems to provide a better yield of cells through culture. However, this requires in vitro culturing to transform these cell lines into alternative mesenchymal cell lines since they have a wide differentiation potential. Although experimental gross osteochondral defect reconstitution and histological grading was superior to periosteum-derived stem cell repair and repair by native mechanisms, this method or same types of approaches still necessitate in vitro tissue culturing (Nathan S, Das De S, Thambyah A, Fen C, Goh J, Lee E H. Cell-based therapy in the repair of osteochondral defects: a novel use for adipose tissue. Tissue Eng. August 2003;9(4):733-44.) Biomechanically, the repair tissue using other sources such as adipose tissue for mesenchymal stem cells can approximate intact cartilage.

Attempts to provide the damaged articular cartilage with a viable durable surface has led to the introduction of soft-tissue grafts consisting of periosteum, perichondrium, fascia, joint capsule and tendinous structures into the defect. Introduced by Rubak in the early 1980's following his experiments with tibial periosteal grafts in rabbit knees, (Rubak J M. Reconstruction of articular cartilage defects with free periosteal grafts: an experimental study. Acta Orthop Scand. 53:175-180, 1982.) this technique appears to be most effective in a younger population. This finding reinforces the notion that age has an adverse effect on the growth and production of pluripotent stem cells and chondrocytes as well as their ability to differentiate into the necessary articular chondrocytes. There is a study that shows that clinically at 10 years follow-up no difference was observed between debridement and drilling and perichondrium transplantation for treatment of an isolated cartilage defect. This raises questions about ongoing research to develop methods in order to improve the results of debridement and drilling as therapy for an isolated cartilage defect in a young patient (Bouwmeester P S, Kuijer R, Homminga G N, Bulstra S K, Geesink R G. A retrospective analysis of two independent prospective cartilage repair studies: autogenous perichondrial grafting versus subchondral drilling 10 years post-surgery. J Orthop Res. March 2002;20(2):267-73.)

A critical component for success with these techniques is that the cambium layer must be placed facing into the joint and the surface must be secured adequately to avoid being knocked loose with joint motion. The potential benefits include the introduction of a new cell population along with an organic matrix, a decrease in the possibility of degeneration of the tissue before a new articular surface can be produced, and an increased protection of the graft from damage due to excessive loading. Periosteal extrusion can cause troublesome mechanical symptoms that might require early revision surgery in patients treated with perichondrial grafting. (Henderson I, Tuy B, Oakes B. Reoperation after autologous chondrocyte implantation. Indications and findings. J Bone Joint Surg Br. March 2004;86(2):205-11.)

The limited ability of chondrocyte cells to effectively differentiate, proliferate, and regenerate hyaline cartilage has increased the interest in of transplanting live cells into chondral defects. This technique consists of injecting the cultivated chondrocytes under a periosteal flap that is sutured over the lesion. Oddly, the technique requires that no penetration of the subchondral bone occur in order to prevent the introduction of blood and the circulating fibrocytes. This technique has received recent widespread attention both in the medical journals and in the media and stimulated patients to request cartilage transplantation Recent research has shown encouraging results regarding the use and efficacy of this technique for focal chondral defects, not for osteoarthritic joints. It is believed that the degradative enzymatic synovial fluid of the arthritic knee is not conducive to cell transfer by this technique. This technique is expensive, does not enable the use of arthroscopic or minimally invasive surgical techniques, requires surgical skills and additional time, and furthermore cannot be performed in a single procedure.

The compositions disclosed herein may be used in any number of tissue repair applications, such as, but not limited to, seroma and hematoma prevention, skin and muscle flap attachment, repair and prevention of endoleaks, aortic dissection repair, lung volume reduction, neural tube repair and the making of microvascular and neural anastomoses.

Further, compositions of the invention may be used as an adhesive composition in the repair of damaged tissue.

In one embodiment, the repair of damaged tissue may be carried out within the context of any standard surgical process allowing access to and repair of the tissue, including open surgery and laparoscopic techniques. Once the damaged tissue is accessed, a composition of the invention is placed in contact with the damaged tissue along with any surgically acceptable patch or implant, if needed. When used to repair lacerated or separated tissue, such as by joining two or more tissue surfaces, the composition may be applied to one or more of the tissue surfaces and then the surfaces are placed in contact with each other and adhesion occurs therebetween.

When used to repair herniated tissue, a surgically acceptable patch can be attached to the area of tissue surrounding the herniated tissue so as to cover the herniated area, thereby reinforcing the damaged tissue and repairing the defect. When attaching the patch to the surrounding tissue, a composition of the invention may be applied to either the patch, to the surrounding tissue, or to the patch after the patch has been placed on the herniated tissue. Once the patch and tissue are brought into contact with each other, adhesion may occur therebetween.

In an embodiment, substantially all reactive components of a composition of the invention are first mixed, then delivered to the desired tissue or surface before substantial cross-linking, for example by electromagnetic radiation, has occurred. The surface or tissue to which the composition has been applied may then contacted with the remaining surface, i.e. another tissue surface or implant surface, preferably immediately, to effect adhesion.

The surfaces to be adhered may be held together manually, or using other appropriate means, while the cross-linking reaction is proceeding to completion. Cross-linking is may typically sufficiently complete for adhesion to occur within about 5 to 60 seconds after mixing the components of the adhesive composition. However, the time required for complete cross-linking to occur is dependent on a number of factors, including the type and molecular weight of each reactive component, the degree of functionalization, and the concentration of the components in the cross-linkable compositions (e.g., higher component concentrations result in faster cross-linking times).

Thus, in one embodiment the compositions of the present invention are delivered to the site of administration using an apparatus that allows the components to be delivered separately. Such delivery systems may involve a multi-compartment spray device.

Alternatively, the components can be delivered separately using any type of controllable extrusion system, or they can be delivered manually in the form of separate pastes, liquids or dry powders, and mixed together manually at the site of administration. Many devices that are adapted for delivery of multi-component tissue sealants/hemostatic agents are well known in the art and can also be used in the practice of the present invention.

Yet another way of delivering the compositions of the present invention is to prepare the reactive components in inactive form as either a liquid or powder. Such compositions can then be activated after application to the tissue site, or immediately beforehand, by applying an activator. In one embodiment, the activator is a buffer solution having a pH that will activate the composition once mixed therewith. Still another way of delivering the compositions is to prepare preformed sheets, and apply the sheets as such to the site of administration. One of skill in the art can easily determine the appropriate administration protocol to use with any particular composition having a known gel strength and gelation time

The compositions described herein can be used for medical conditions that require a coating or sealing layer to prevent the leakage of gases, liquid or solids. The method entails applying both components to the damaged tissue or organ to seal 1) vascular and or other tissues or organs to stop or minimize the flow of blood; 2) thoracic tissue to stop or minimize the leakage of air; 3) gastrointestinal tract or pancreatic tissue to stop or minimize the leakage of fecal or tissue contents; 4) bladder or ureters to stop or minimize the leakage of urine; 5) dura to stop or minimize the leakage of CSF; and 6) skin or serosal tissue to stop the leakage of serosal fluid. These compositions may also be used to adhere tissues together such as small vessels, nerves or dermal tissue. The material can be used 1) by applying it to the surface of one tissue and then a second tissue maybe rapidly pressed against the first tissue or 2) by bringing the tissues in close juxtaposition and then applying the material. In addition, the compositions can be used to fill spaces in soft and hard tissues that are created by disease or surgery.

For example, polymer matrix compositions of the invention can be used to block or fill various lumens and voids in the body of a mammalian subject. The compositions can also be used as biosealants to seal fissures or crevices within a tissue or structure (such as a vessel), or junctures between adjacent tissues or structures, to prevent leakage of blood or other biological fluids.

The compositions can also be used as a large space-filling device for organ displacement in a body cavity during surgical or radiation procedures, for example, to protect the intestines during a planned course of radiation to the pelvis.

The compositions of the invention can also be coated onto the interior surface of a physiological lumen, such as a blood vessel or Fallopian tube, thereby serving as a sealant to prevent restenosis of the lumen following medical treatment, such as, for example, balloon catheterization to remove arterial plaque deposits from the interior surface of a blood vessel, or removal of scar tissue or endometrial tissue from the interior of a Fallopian tube. A thin layer of the reaction mixture is preferably applied to the interior surface of the vessel (for example, via catheter) immediately following mixing of the first and second synthetic polymers. Because the compositions of the invention are not readily degradable in vivo, the potential for restenosis due to degradation of the coating is minimized.

The compositions of the invention can also be used for augmentation of soft or hard tissue within the body of a mammalian subject. Examples of soft tissue augmentation applications include sphincter (e.g., urinary, anal, esophageal) augmentation and the treatment of rhytids and scars. Examples of hard tissue augmentation applications include the repair and/or replacement of bone and/or cartilaginous tissue.

The compositions of the invention may be used as a replacement material for synovial fluid in osteoarthritic joints. The compositions may reduce joint pain and improve joint function by restoring a soft gel network in the joint. The crosslinked polymer compositions can also be used as a replacement material for the nucleus pulposus of a damaged intervertebral disk. The nucleus pulposus of the damaged disk is first removed, and the reactive composition is then injected or otherwise introduced into the center of the disk. The composition may either be cross-linked prior to introduction into the disk, or allowed to cross-link in situ.

In some embodiments, one, two, or more polymerizing agents may be used. For example, electromagnetic radiation may be used alone, or together with a photoinitiator. A photoinitiator alone may be used. Additionally or independently, a redox polymerizing agent may be used. The electromagnetic radiation, or a photoinitiator may trigger a fast polymerization. Such fast polymerization may ensure that the composition remains in the desired location. A redox polymerizing agent may be used simultaneously, before, or after electromagnetic radiation. A redox polymerizing agent may trigger a slow polymerization, for example, about 2 hours.

In a general method for effecting augmentation of tissue or a disk within the body of a mammalian subject, the components of the reactive composition are injected, implanted, or infused simultaneously to a tissue or disk site in need of augmentation. The present invention may be prepared to include an appropriate vehicle for this injection, implantation, infusion or direction. Once inside the patient's body, the functionalized chondroitin sulfate and, for example, a compound comprising an amine group may react with each other to form a crosslinked polymer network in situ. The functionalized chondroitin sulfate may also react with primary amino groups on, for example, lysine residues collagen molecules within the patient's own tissue, providing for “biological anchoring” of the compositions with the host tissue.

The polymer matrix, alternatively, may be formed as a solid object implantable in the anatomic area, or as a film or mesh that may be used to cover a segment of the area A variety of techniques for implanting solid objects in relevant anatomic areas will be likewise familiar to practitioners of ordinary skill in the art.

In some embodiments, compositions disclosed herein may be positioned in a surgically created defect that is to be reconstructed, and is to be left in this position after the reconstruction has been carried out. The present invention may be suitable for use with local tissue reconstructions, pedicle flap reconstructions or free flap reconstructions.

In some embodiments, this invention is directed to assays and kits for assessing effectiveness and diagnosis of cartilage degradation diseases such as arthritis. In some embodiments, the assay or kits detect the presence of enzymes that may degrade a cross-linked polymer matrix of this disclosure.

Test kits for use may include cross-linked matrix polymers comprising functionalized disaccharides that degrade in the presence of cartilage degrading enzymes, for example, chondroitinase and collagenase. Other proteases and enzymes may be detected using such kits.

The invention will now be described in the following non-limiting examples.

### Example 1

### Materials

Chondroitin sulfate A sodium salt (CS, Type A 70%, balanced with Type C from bovine trachea) and Acetone (<0.5% water) is obtained from SIGMA, MO. Glycidyl methacrylate (GMA, 98% purity) is obtained from Polysciences, PA. Acrylate-PEG-Acrylate (PEODA, 100% M 3127, Polydispersity=1.03, as determined by GPC analysis) is obtained from Shearwater, AL. Phosphate saline buffer (PBS, pH7.4) may be obtained from GEBCO.

### Example 2

### Synthesis of GMA-CS

Ten grams of CS is dissolved in 100 ml PBS, followed by addition of 10 ml GMA, while vigorously stirring at room temperature. Samples are collected at Days 1,3, 5,7, 10 and 15 by acetone precipitation and purified twice by acetone extraction. The GMA-CS products (Day 1,3, 5,7, 10 and 15) are lyophilized for 24 hrs and stored at 4 C.

### Example 3

### Synthesis of Aldehyde Functionalized CS and Cross-Linked Matrix

Six hundred mg of chondroitin sulfate Type A (0.8-1.2 mmol of adjacent diol, 70% CS-A, Sigma) and 616 mg of sodium periodate (−2.88 mmol, NaIO4, Sigma) are dissolved together in 10 ml of de-ionized water and protected from light. The reaction is allowed to continue for-14 hr in dark with vigorous stirring. The insoluble byproducts are removed with 0.22 um filter and the product is loaded into a Sephadex G-25 (Sigma) size exclusion chromatography (SEC) column, by which the product was purified from the water-soluble byproducts and un-reacted small molecules. The product, chondroitin sulfate-aldehyde (CS-ald), is obtained by lyophilization with a yield rate of-90%. The determination of aldehyde substitution degree is performed via a hydroxylamine hydrochloride titration. The result is 60-70% substitution.

A tissue adhesive is formulated by mixing equal volumes (20 ml) of 25% CS-ald and 40% bovine serum albumin (BSA, Sigma). The adhesive is used immediately after the formulation and the reaction is completed in 2-5 min with the Schiff-base mechanism.

### Example 4

### NMR Methods

NMR spectra are recorded with a Unity Plus 500 MHz spectrometer (Varian Associates). For H-NMR in deuterium-d2 (D20,99. 9% h, SIGMA) approximately 50 mg material was dissolved in 1.0 ml D20, and 2HOH at 4.8 ppm was used as the reference peak.

For 13C-NMR in deuterium-d2 (D20, 99.9% 2H, SIGMA) the pulse is 51.9 degrees, using a pulse length of 7 ps, acquisition time of 1.300 sec, and 80000 repetitions at 50 C.

### Example 5

### Photocrosslinking and Hydrogel Swelling Ratio

GMA-CS and PEODA are mixed 1:1 (w/w) and dissolved in water for a GMA-CS concentration of 10% (w/w). One hundred fifty liters of macromer solution. (10% w/v)) are placed in tissue insert (diameter 8 mm) and polymerized. Photocrosslinking is initiated with a cytocompatible W photoinitiator Ingracure 2959 (0.05% w/w, Ciba Geigy) and 365 nm light at-10 mW/cm2 as measured by a radiometer. The macromers are photopolymerized for 30 min.

The photocross-linked hydrogels are equilibrated in PBS at 37 C for 18 h. The water content of the hydrogels is determined by measuring the wet weight (Ww) of the constructs. Dry weight (Wd) of the hydrogels was measured after lyophilization for 24 h.

The hydrogel equilibrated swelling ratio, q, is calculated by qz Ww/Wd.

### Example 6

### Rheological Characterization

PBS-equilibrated copolymerized CS-MA and poly (ethylene oxide)-diacrylate (PEODA) (3,400; Shearwater Polymers, Knoxville, Tenn.) macromers (20% w/v) hydrogel constructs are prepared in tissue culture inserts as previously described. The constructs average 13.21±0.86 mm in diameter and 4.67±0.16 mm in thickness as measured by current sensing micrometer. The weight percentage of PEODA and CS-MA in the constructs is varied from 0% (i.e., pure PEODA), 25%, 50%, 75% and 100% (i.e., pure CS-MA).

Rheological tests are performed on a RFS-3 rheometer (Rheometric Scientific Inc.) using the parallel-plate configuration. The pilot dynamic shear strain-sweep test at a frequency 6.28 rad/s indicates a 0.1% shear strain that is in the linear stress-strain range for the samples with various concentration ratios, and such linearity is confirmed using the dynamic shear strain-sweep test for each test sample prior to the dynamic shear frequency-sweep test. The dynamic shear frequency-sweep is tested over a range of frequencies from 0.1 to 100 rad/s at a shear amplitude of 0.1%.

### Example 7

### Morphological Analysis

Hydrogel blocks synthesized from 20% (w/v) macromer solutions of CS-MA and PEODA were cut, frozen, and lyophilized. The surface and the cut edge of the hydrogels are analyzed on a LEO 1530 Field Emission scanning electron microscope (LEO Electron Microscopy Inc.).

### Example 8

### Degradation Experiments

Degradation of the polymerized hydrogels is carried out in pH 8.0 Tris-HCl buffered digestion solution (Tris-HCl 60 mM/L, sodium acetate 40 mM/L and bovine serum albumin 1.5×10-4 mg/L) at 37 C, 5% C02. Photopolymerized CS-MA hydrogels (20% w/v) are weighed and placed in 24-well cell culture plate with 2.5 ml digestion buffer with or without chondroitinase ABC (0.8 mg/ml). At specified time points, the weights of constructs are measured. Chondroitinase ABC concentration is also varied (0.0025 g/ml, 0.025 g/ml, 0.25 g/ml and 2.5 g/ml) and at specified time points, the absorbance of digestion solutions is measured at 232 nm with a background subtraction at 600 nm in order to monitor disaccharide evolution as degradation proceeded (n−3). Values are normalized to hydrogel construct original weight. The gels are completely degraded by 33 hours in the presence of enzyme compared to control gels incubated without enzymes that maintain a constant weight throughout the experiment. Release of degraded chondroitin sulfate from the gels was measured in the buffer with varying concentrations of chondoitinase enzyme. Increasing the enzyme concentration increases the concentration of degradation byproducts observed in the surrounding buffer.

### Example 9

### Cell Encapsulation and Viability

CS-MA and PEODA are combined in a 1:1 ratio and dissolved in PBS with 100 U/ml penicillin G and 100 ug/ml streptomycin to from a 20% (w/v) solution. After addition of 0.05% Irgacure D-2959 (w/v), the macromer solutions are added to re-suspend the cell pellet to make a final concentration of 20×106 cells/ml, and subsequently photopolymerized for 8 min with 10 mW/cm2 UV light. The constructs are then transferred and incubated in chondrocyte media high-glucose Dulbecco's modified Eagle's medium (DMEM), 10% fetal bovine serum (FBS), 10, ug/ml vitamin C, 12.5 mM HEPES, 0.1 mM nonessential amino acids and 0.4 mM proline] at 37 C, 5% C02.

MTT assay and live/dead staining assay are respectively performed to measure cell viability after 1 day in culture. For MTT assay, the constructs are washed twice with PBS and 2 mls of MTT solution (0.5 mg/ml in DMEM with 2% FBS) are added to each well for 2-4 h. Actively metabolizing cells are observed by light microscopy. Cell viability of the encapsulated cells is also evaluated with Live/Dead Viability/Cytotoxicity Kit (Molecular Probes, Eugene, Oreg., U.S.A.). Thin slices (100-200 um) of three layers are prepared with a surgical blade from the constructs. The slices are incubated for 30 minutes in Live/Dead assay reagents (2 uM calcein AM and 4 AM. Fluorescence microscopy is performed using a fluorescein optical filter (485±10 nm) for calcein AM and a rhodamine optical filter (530 12.5 nm) for Ethidium homodimer-1.

### Example 10

### IVD Applications

A polymer composition with 80% CSMA with 0.1% (w/v) Irgacure D2959 photoinitiator or a polymer composition with 50% CSMA/10% PEODA with 0.1% (w/v) Irgacure D2959 is used. Gels photopolymerized in a IVD space in part A are removed and the swelling ratio is determined. A water-soluble redox initiating system is used with CSMA that includes 0.1% D2959 and 0.15 M sodium persulfate-0.12M sodium thiosulfate.

The system is implanted in cadaveric IVD space. After photopolymerization the cadaveric spine is be placed in a 37 C incubator to allow the redox polymerization. After gelation, the gel size and water content is determined. Results obtained in this model are expected to correlate with in vivo results.

### Example 11

### Rabbit Studies

An IVD rabbit stab model is used to mimic the normal disc degeneration process. Animals are anesthetized with 50 mg/kg ketamine IM and 10 mg/kg xylazine IM and a stab wound is created in the IVD disk space using an 18-gauge needle. Discs are allowed to degenerate for four weeks before polymer injection. The polymer formulation is injected into the disrupted disk space and polymerized. Control IVD disc spaces are injected with saline instead of polymer. Animals are monitored radiographically once a week to observe implant placement, disk height, and tissue degradation or inflammation. Animals are sacrificed after 4,8 and 12 weeks and histological analysis is performed to observe polymer size and shape, inflammation, and surrounding tissue integration and repair.

All references cited herein are herein incorporated by reference in entirety.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A biocompatible polymer comprising at least two functional groups.

2. The polymer of claim 1, wherein said polymer is a natural polymer.

3. The polymer of claim 2, wherein said polymer is chondroitin sulfate.

4. The polymer of claim 1, wherein said polymer is a synthetic polymer.

5. The polymer of claim 2, wherein said natural polymer is a component of a tissue.

6. The polymer of claim 1, wherein said at least two functional groups are the same.

7. The polymer of claim 1, wherein one of said at least two functional groups reacts with a tissue.

8. A composition comprising the polymer of claim 1 and a hydrogel.

9. The composition of claim 8, wherein one of said at least two functional groups of said polymer reacts with said hydrogel.

10. The composition of claim 9, wherein the second of said at least two functional groups reacts with a tissue.

11. The composition of claim 8, further comprising cells.

12. The composition of claim 8, wherein said hydrogel comprises polyethylene oxide diacrylate.

13. The composition of claim 8, wherein said hydrogel comprises a viscous liquid.

14. The composition of claim 8, wherein said hydrogel is produced by photopolymerization.

15. The composition of claim 13, wherein said viscous liquid comprises a hyaluronic acid or a modified hyaluronic acid.

16. The composition of claim 8, wherein one of said at least two functional groups of said polymer is an acrylate group.

17. The composition of claim 8, further comprising a biologically active agent.
